(19) United States
(12) Patent Application Publication
(10) Pub. No.: US 2015/0203444 A1
(43) Pub. Date: Jul. 23, 2015

(54) POLYMORPHS OF N-((S)-3-AMINO-1-(HYDROXYAMINO)-3-METHYL-1-OXOBUTAN-2-YL)-4-(((1R,2R)-2-(HYDROXYMETHYL)CYCLOPROPYL)BUTA-1,3-DIYNYL)BENZAMIDE

(71) Applicants: Raissa Trend, San Francisco, CA (US); Ramesh Annasaheb Kasar, Oakland, CA (US)

(72) Inventors: Raissa Trend, San Francisco, CA (US); Ramesh Annasaheb Kasar, Oakland, CA (US)

(73) Assignee: Achaogen, Inc., South San Francisco, CA (US)

(21) Appl. No.: 13/611,149

(22) Filed: Sep. 12, 2012

Related U.S. Application Data
(60) Provisional application No. 61/533,628, filed on Sep. 12, 2011.

(51) Int. Cl. C07C 235/42

(52) U.S. Cl. CPC C07C 235/42; C07B 2200/13

(57) ABSTRACT

Polymorphs of N—((S)-3-amino-1-(hydroxyamino)-3-methyl-1-oxobutan-2-yl)-4-(((1R,2R)-2-(hydroxymethyl)cyclopropyl)buta-1,3-diynyl)benzamide (Compound I) are provided. Processes for making the same, as well as related compositions and methods, are also disclosed, particularly with regard to the treatment of bacterial infections.

### CROSS-REFERENCE TO RELATED APPLICATIONS

This application claims the benefit under 35 U.S.C. §119(e) of U.S. Provisional Patent Application No. 61/533,628, filed Sep. 12, 2011. The foregoing application is incorporated herein by reference in its entirety.

### STATEMENT OF GOVERNMENT INTEREST

This invention was made with government support under Contract No. HDTRA1-07-C-0079 awarded by the United States Department of Defense. The government has certain rights in this invention.

### BACKGROUND

1. Field

This invention is directed to polymorphs of N—((S)-3-amino-1-(hydroxyamino)-3-methyl-1-oxobutan-2-yl)-4-(((1R,2R)-2-(hydroxymethyl)cyclopropyl)buta-1,3-diynyl)benzamide (Compound I) having activity as antibacterial agents, particularly in the treatment of infections caused by gram-negative bacteria by inhibiting activity of UDP-3-O—(R-3-hydroxydecanoyl)-N-acetylglucosamine deacetylase (LpxC), and to related processes, compositions and methods.

2. Description of the Related Art

Over the past several decades, the frequency of antimicrobial resistance and its association with serious infectious diseases have increased at alarming rates. The problem of antibacterial resistance is compounded by the existence of bacterial strains resistant to multiple antibacterials. Thus there is a need for new antibacterials, particularly antibacterials with novel mechanisms of action. A previously unexploited but highly conserved target, LpxC, provides a new opportunity for developing broad-spectrum antibacterial small molecules that comprise a new class of active bactericidal chemical entities that should encounter little, if any, naturally-occurring, target-related resistance. LpxC (the enzyme uridyldiphospho-3-O—(R-hydroxydecanoyl)-N-acetylglucosamine deacetylase) is present across all Gram-negative bacterial species of interest and is involved in the first committed step in outer membrane biosynthesis. Thus LpxC is essential for survival and presents an ideal target for antibiotic activity in Gram-negative bacterial species.

Researchers have identified some compounds with antibacterial activity that target lipid A biosynthesis. For example, Jackman et al. (J. Biol. Chem., 2000, 275(15), 11002-11009); Wyckoff et al. (Trends in Microbiology, 1998, 6(4), 154-159); U.S. Patent Application Publication No. 2001/0053555 (published 20 Dec. 2001, corresponding to International PCT Publication No. WO 98/18754, published 7 May 1998); International PCT Publication No. WO 00/61134 (published 19 Oct. 2000); U.S. Patent Application Publication No. 2004/0229955 (published 18 Nov. 2004); and International PCT Publication No. WO 2008/154642 (published 18 Dec. 2008) all disclose compounds having antibacterial anti-LpxC activity. However, the commercial development of these LpxC inhibitors has been complicated by toxicity of these compounds in mammalian animals at concentrations at or near those required for antibacterial activity.

More recently, as disclosed in co-pending U.S. Provisional Patent Application No. 61/412,311 (filed 10 Nov. 2010), compounds having anti-LpxC activity which are significantly better tolerated than other closely related compounds have been identified. In particular, N—((S)-3-amino-1-(hydroxyamino)-3-methyl-1-oxobutan-2-yl)-4-(((1R,2R)-2-(hydroxymethyl)cyclopropyl)buta-1,3-diynyl)benzamide (Compound 1) has been identified as a particularly active and well tolerated compound.

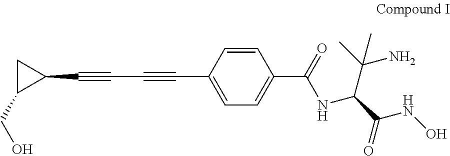 [SMILES: CC(C)(N)[C@H](NC(=O)c1ccc(C#CC#C[C@@H]2C[C@H]2CO)cc1)C(=O)NO]

Accordingly, although there have been advances in the field, there remains a need for LpxC inhibitors that have activity as bactericidal agents against gram-negative bacteria and have an acceptable toxicity/tolerance profile. In addition, the remains a need for improved forms of such compounds, particularly with regard to enhanced solubility, oral bioavailability and/or physical stability. The present invention fulfills one or more of these needs and provides further related advantages.

### BRIEF SUMMARY

This invention is directed to novel polymorphs of N—((S)-3-amino-1-(hydroxyamino)-3-methyl-1-oxobutan-2-yl)-4-(((1R,2R)-2-(hydroxymethyl)cyclopropyl)buta-1,3-diynyl)benzamide (Compound I) (referred to herein as “Form A”, “Form B” and “Form C”). Polymorph Forms A, B and C have activity as antibacterial agents, particularly in the treatment of infections caused by gram-negative bacteria by inhibiting activity of UDP-3-O—(R-3-hydroxydecanoyl)-N-acetylglucosamine deacetylase (LpxC).

Polymorph Form A exhibits an X-ray Powder Diffraction pattern with characteristic peaks (expressed in degrees 2θ (+/−0.208)) at one or more of the following positions: 5.1, 5.5 and 6.3. Polymorph Form A also exhibits three predominant endotherm peaks at about 87° C., about 115° C. and about 124° C. as (as measured by a Mettler 822e Differential Scanning Calorimeter (DSC) at a scan rate of 10° C. per minute.

Polymorph Form B exhibits an X-ray Powder Diffraction pattern with characteristic peaks (expressed in degrees 2θ (+/−0.2°θ)) at one or more of the following positions: 18.0, 20.0, 21.1, 22.3 and 24.9. Polymorph Form B also exhibits a predominant endotherm peak at about 168° C. (as measured by a Mettler 822e Differential Scanning Calorimeter (DSC) at a scan rate of 10° C. per minute.

Polymorph Form C exhibits an X-ray Powder Diffraction pattern with a characteristic peak (expressed in degrees 2θ (+/−0.2°θ)) a 8.0. Polymorph Form C also exhibits a predominant endotherm peak at about 125° C. (as measured by a Mettler 822e Differential Scanning Calorimeter (DSC) at a scan rate of 10° C. per minute.

The present invention is also directed to pharmaceutical compositions comprising a pharmaceutically acceptable carrier or diluent and polymorph Form A, Form B or Form C.

In other embodiments, the present invention is directed to a method for treating a subject having a bacterial infection comprising administering to a subject in need thereof a therapeutically effective amount of polymorph Form A, Form B or Form C. In certain embodiments, said bacterial infection is a gram-negative bacterial infection. In certain embodiments, said gram-negative bacterial infection is Pseudomonas aeruginosa, Stenotrophomonas maltophila, Burkholderia cepacia, Alcaligenes xylosoxidans, or a Enterobacteriaceae, Haemophilus, Franciscellaceae or Neisseria species. In certain embodiments, said gram-negative bacteria is a member of the Enterobacteriaceae selected from the group consisting of Serratia, Proteus, Klebsiella, Enterobacter, Citrobacter, Salmonella, Providencia, Yersinia, Morganella, Cedecea, Edwardsiella and Escherichia.

In other embodiments, the present invention is directed to a method of inhibiting a deacetylase enzyme in gram-negative bacteria comprising administering to a subject in need of such inhibition polymorph Form A, Form B or Form C. In certain embodiments, the gram-negative bacteria are Pseudomonas aeruginosa, Stenotrophomonas maltophila, Burkholderia cepacia, Alcaligenes xylosoxidans, or a Enterobacteriaceae, Haemophilus, Franciscellaceae, or Neisseria species.

In other embodiments, the present invention is directed to a method of inhibiting LpxC comprising administering to a subject in need of such inhibition an effective amount of polymorph Form A, Form B or Form C.

These and other aspects of the invention will be evident upon reference to the following detailed description.

### BRIEF DESCRIPTION OF THE DRAWINGS

FIG. 1 is a Differential Scanning Calorimetry (DSC) thermogram of polymorph Form A.

FIG. 2 is a Differential Scanning Calorimetry (DSC) thermogram of polymorph Form B.

FIG. 3 is a Differential Scanning Calorimetry (DSC) thermogram of polymorph Form C.

FIG. 4 is an X-ray powder diffraction spectrum of polymorph Form A.

FIG. 5 is an X-ray powder diffraction spectrum of polymorph Form B.

FIG. 6 is an X-ray powder diffraction spectrum of polymorph Form C.

FIG. 7 is a stack plot of the X-ray powder diffraction patterns of polymorph Forms A, B and C.

FIG. 8 is a Raman FT Infrared spectrum of polymorph Form A.

FIG. 9 is a Raman FT Infrared spectrum of polymorph Form B.

FIG. 10 is a Raman FT Infrared spectrum of polymorph Form C.

FIG. 11 is a stack plot of the Raman FT Infrared spectrum of polymorph Forms A, B and C.

### DETAILED DESCRIPTION

Unless the context requires otherwise, throughout the specification and claims which follow, the word “comprise” and variations thereof, such as, “comprises” and “comprising” are to be construed in an open, inclusive sense, that is as “including, but not limited to”.

Reference throughout this specification to “one embodiment” or “an embodiment” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used herein, “pharmaceutically effective amount” and “therapeutically effective amount” refer to an amount of a polymorph sufficient to treat a specified disorder or disease or one or more of its symptoms and/or to prevent the occurrence of the disease or disorder. The term “antibacterial agent” refers to agents that have either bactericidal or bacteriostatic activity. The term “inhibiting the growth” indicates that the rate of increase in the numbers of a population of a particular bacterium is reduced. Thus, the term includes situations in which the bacterial population increases but at a reduced rate, as well as situations where the growth of the population is stopped, as well as situations where the numbers of the bacteria in the population are reduced or the population even eliminated. If an enzyme activity assay is used to screen for inhibitors, one can make modifications in uptake/efflux, solubility, half-life, etc. to compounds in order to correlate enzyme inhibition with growth inhibition. The activity of antibacterial agents is not necessarily limited to bacteria but may also encompass activity against parasites, virus, and fungi.

As mentioned above, the present invention is directed to novel polymorphic form of N—((S)-3-amino-1-(hydroxyamino)-3-methyl-1-oxobutan-2-yl)-4-(((1R,2R)-2-(hydroxymethyl)cyclopropyl)buta-1,3-diynyl)benzamide (Compound I) (referred to herein as “Form A”, “Form B” and “Form C”), as well as to compositions containing the same. Also disclosed are methods relating to the use of such polymorphs by administration to a subject in need of the same, and to processes for making such polymorphs.

Solids exist in either amorphous or crystalline forms. In the case of crystalline forms, molecules are positioned in 3-dimensional lattice sites. When a compound recrystallizes from a solution or slurry, it may crystallize with different spatial lattice arrangements, a property referred to as “polymorphism,” with the different crystal forms individually being referred to as a “polymorph”. Different polymorphic forms of given substance may differ from each other with respect to one or more physical properties, such as solubility and dissociation, true density, crystal shape, compaction behavior, flow properties, and/or solid state stability. In the case of a chemical substance that exists in two (or more) polymorphic forms, the unstable forms generally convert to the more thermodynamically stable forms at a given temperature after a sufficient period of time. When this transformation is not rapid, the thermodynamically unstable form is referred to as the “metastable” form. In general, the stable form exhibits the highest melting point, the lowest solubility, and the maximum chemical stability. However, the metastable form may exhibit sufficient chemical and physical stability under normal storage conditions to permit its use in a commercial form. In this case, the metastable form, although less stable, may exhibit properties desirable over those of the stable form, such as enhanced solubility or better oral bioavailability.

In the practice of this invention, three different polymorphs (Forms A, B and C) of N—((S)-3-amino-1-(hydroxyamino)-3-methyl-1-oxobutan-2-yl)-4-(((1R,2R)-2-(hydroxymethyl)cyclopropyl)buta-1,3-diynyl)benzamide (Compound I) have been discovered. Form A is a semi-crystalline, moderately hygroscopic solid. Form B is a crystalline solid and appears to be the most thermodynamically stable of the three polymorphic forms. Form B also exhibited stability to prolonged thermal stress and exposure to elevated relative humidity. Form C is a metastable polymorph.

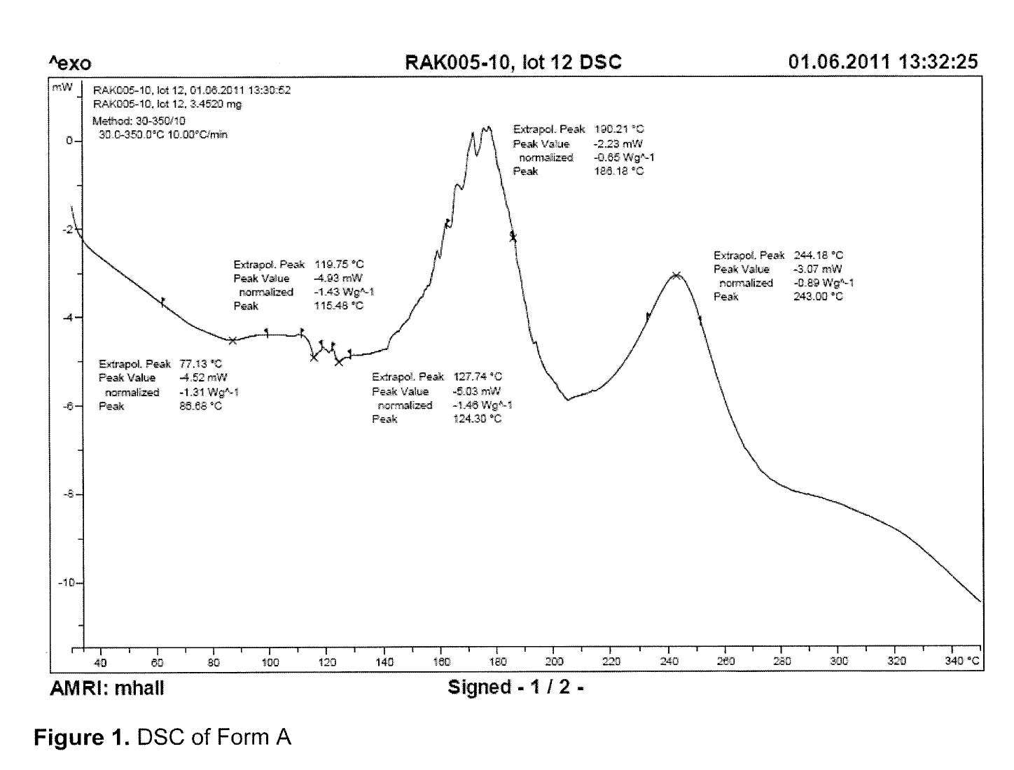

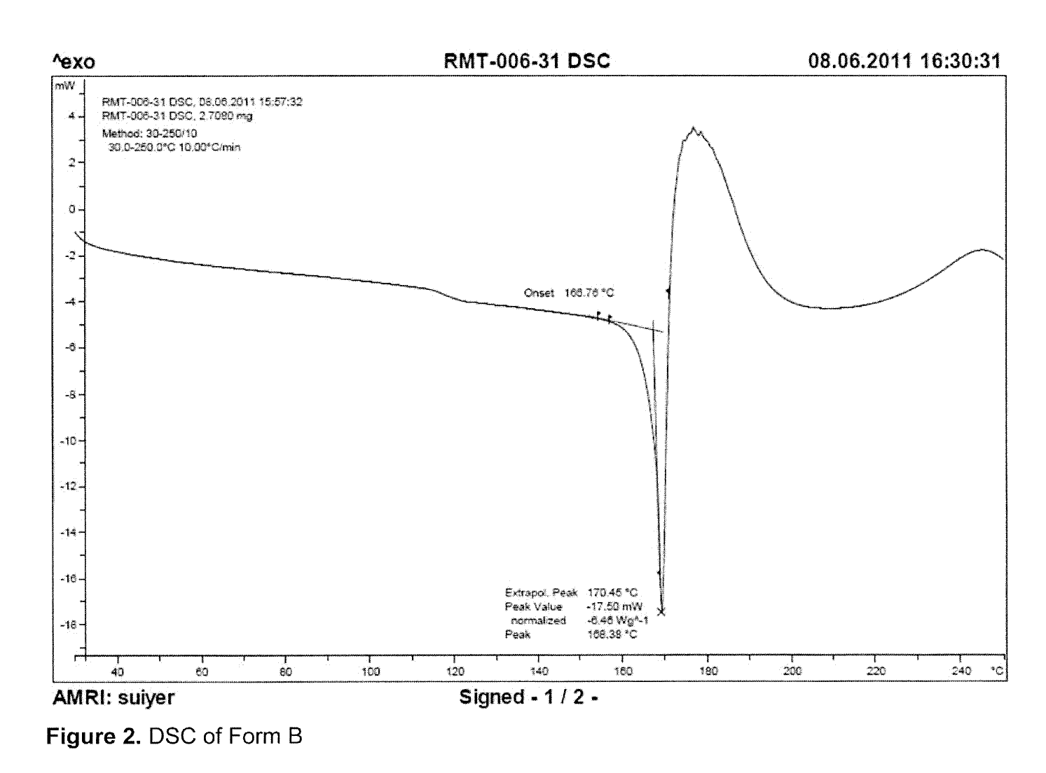

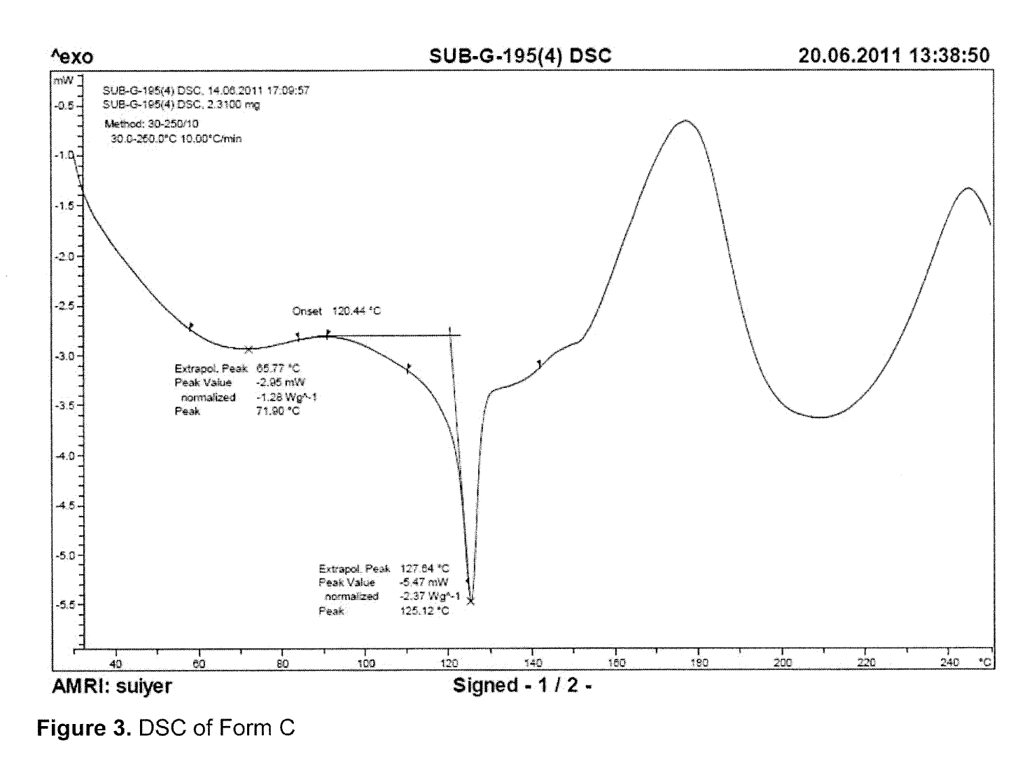

The novel polymorphs of this invention, polymorph Forms A, B and C, may be characterized by, for example, melting point and/or X-Ray powder diffraction spectrometry. As shown in FIG. 1, polymorph Form A exhibits three predominant endothermic peaks at about 87° C., about 115° C. and about 124° C. as measured by a Mettler 822e (Mettler Toledo) Differential Scanning Calorimeter (DSC) at a scan rate of 10° C. per minute. As shown in FIG. 2, polymorph Form B exhibits a predominant endotherm peak at about 168° C. as measured by a Mettler 822e (Mettler Toledo) Differential Scanning Calorimeter (DSC) at a scan rate of 10° C. per minute. As shown in FIG. 3, polymorph Form C exhibits a predominant endotherm peak at about 125° C. as measured by a Mettler 822e (Mettler Toledo) Differential Scanning Calorimeter (DSC) at a scan rate of 10° C. per minute.

Depending upon the rate of heating, i.e. the scan rate, at which the DSC analysis is conducted, the calibration standard used, instrument calibration, the relative humidity and upon the chemical purity, the endotherms of the various polymorphic Forms may vary by about 0.01-10° C., or about 0-5° C., above or below the endotherms depicted in the drawings. For any given sample, the observed endotherm may also differ from instrument to instrument; however, it will generally be within the ranges defined herein provided the instruments are calibrated similarly.

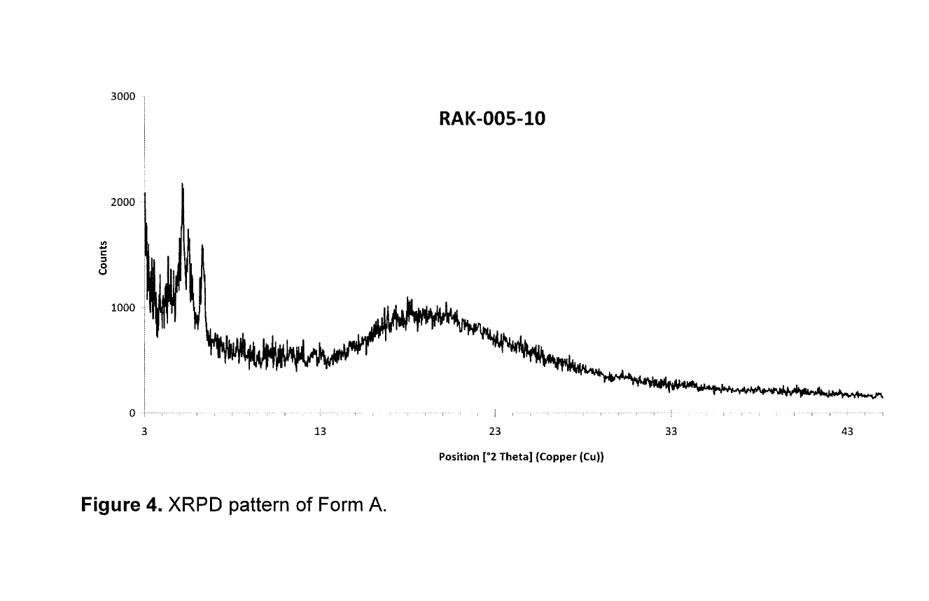

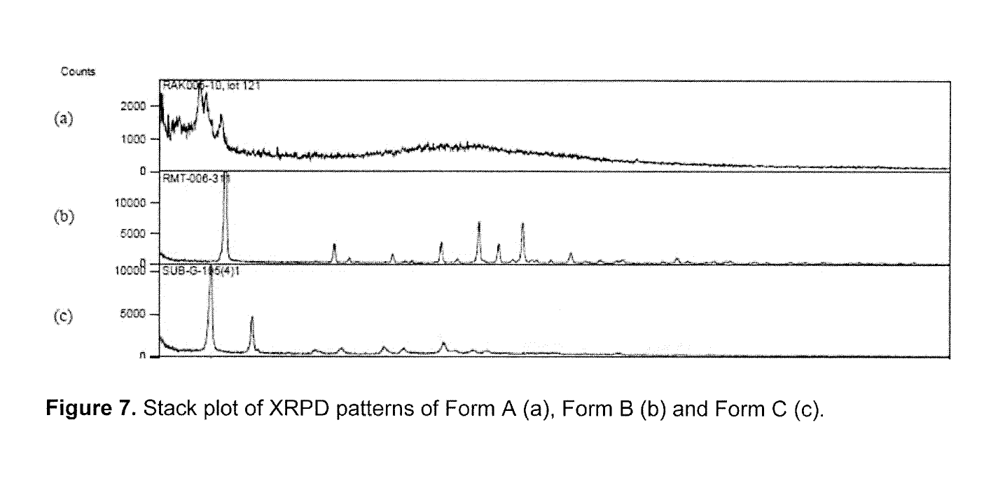

The X-ray powder diffraction spectrum for polymorph Forms A, B and C are presented in FIGS. 4, 5 and 6, respectively, and are set forth in tabular form in Tables 1, 2 and 3, respectively, below. In addition, a stack plot of the XRPD patterns of polymorph Forms A, B and C is presented in FIG. 7. The X-ray powder diffraction was measured by a PANalytical Cubix Pro X-ray Powder Diffractometer. Analysis was performed using a 10 mm irradiated width and the following parameters were set within the hardware/software:
X-ray tube: Cu KV, 45 kV, 40 mA
Detector: X'Celerator
ASS Primary Slit: Fixed 1°
Divergence Slit (Prog): Automatic—5 mm irradiated length
Soller Slits: 0.02 radian
Scatter Slit (PASS): Automatic—5 mm observed length
Scan Range: 3.0-45.0°
Scan Mode: Continuous
Step Size: 0.02°
Time per Step: 10 s
Active Length: 2.54°
   Following analysis the data was converted from adjustable to fixed slits using the X'Pert HighScore Plus software with the following parameters:
Fixed Divergence Slit Size: 1.000, 1.59 mm
Crossover Point: 44.3°

TABLE 1
Polymorph Form A
X-Ray Powder Diffraction Spectral Lines

| d value | 2-θ° | Intensity | Intensity % |
|---|---|---|---|
| 21.65114 | 4.0812 | 1251.96 | 56.24 |
| 17.28936 | 5.1114 | 2225.95 | 100.00 |
| 16.03258 | 5.5123 | 1956.85 | 87.91 |
| 15.26266 | 5.7906 | 1065.53 | 47.87 |
| 14.02822 | 6.3007 | 1261.29 | 56.66 |
| 13.46269 | 6.5656 | 568.02 | 25.52 |
| 11.40651 | 7.7509 | 154.32 | 6.93 |
| 11.05837 | 7.9953 | 143.22 | 6.43 |
| 10.38362 | 8.5157 | 202.89 | 9.11 |
| 9.53709 | 9.2732 | 154.08 | 6.92 |
| 5.26988 | 16.8241 | 210.49 | 9.46 |
| 4.90331 | 18.0921 | 172.37 | 7.74 |
| 4.48199 | 19.8091 | 164.25 | 7.38 |
| 3.14735 | 28.3576 | 87.24 | 3.92 |

TABLE 2
Polymorph Form B
X-Ray Powder Diffraction Spectral Lines

| d value | 2-θ° | Intensity | Intensity % |
|---|---|---|---|
| 28.15119 | 3.1386 | 1338.01 | 8.99 |
| 24.35295 | 3.6282 | 613.42 | 4.12 |
| 22.35492 | 3.9526 | 514.82 | 3.46 |
| 20.22674 | 4.3687 | 635.89 | 4.27 |
| 18.75036 | 4.7129 | 454.47 | 3.05 |
| 18.07327 | 4.8895 | 535.98 | 3.60 |
| 16.15163 | 5.4717 | 484.61 | 3.26 |
| 15.21970 | 5.8070 | 361.94 | 2.43 |
| 13.66199 | 6.4698 | 14885.51 | 100.00 |
| 13.40377 | 6.5945 | 14877.59 | 99.95 |
| 10.63422 | 8.3147 | 283.25 | 1.90 |
| 10.02957 | 8.8169 | 307.98 | 2.07 |
| 7.77908 | 11.3751 | 272.89 | 1.83 |
| 7.18528 | 12.3187 | 3081.63 | 20.70 |
| 6.74707 | 13.1222 | 876.64 | 5.89 |
| 6.54702 | 13.5250 | 341.34 | 2.29 |
| 5.89206 | 15.0367 | 205.15 | 1.38 |
| 5.74858 | 15.4142 | 1544.30 | 10.37 |
| 5.51751 | 16.0639 | 467.40 | 3.14 |
| 5.38624 | 16.4581 | 460.37 | 3.09 |
| 5.16854 | 17.1565 | 124.28 | 0.83 |
| 4.92491 | 18.0120 | 3424.84 | 23.01 |
| 4.69828 | 18.8886 | 749.61 | 5.04 |
| 4.43763 | 20.0092 | 6868.53 | 46.14 |
| 4.37059 | 20.3193 | 470.13 | 3.16 |
| 4.21740 | 21.0657 | 3290.94 | 22.11 |
| 4.07683 | 21.8008 | 683.55 | 4.59 |
| 3.98152 | 22.3293 | 6630.67 | 44.54 |
| 3.89821 | 22.8128 | 598.94 | 4.02 |
| 3.85660 | 23.0623 | 554.75 | 3.73 |
| 3.73729 | 23.8092 | 539.43 | 3.62 |
| 3.58040 | 24.8688 | 1758.22 | 11.81 |
| 3.47719 | 25.6193 | 470.97 | 3.16 |
| 3.37045 | 26.4451 | 572.07 | 3.84 |
| 3.27077 | 27.2663 | 426.39 | 2.86 |
| 3.22992 | 27.6180 | 594.25 | 3.99 |
| 3.09954 | 28.8043 | 106.16 | 0.71 |
| 3.00205 | 29.7609 | 371.51 | 2.50 |
| 2.93123 | 30.4972 | 895.08 | 6.01 |
| 2.87831 | 31.0719 | 479.61 | 3.22 |
| 2.79109 | 32.0688 | 206.37 | 1.39 |
| 2.75754 | 32.4696 | 399.59 | 2.68 |
| 2.70391 | 33.1319 | 354.74 | 2.38 |
| 2.68642 | 33.3540 | 421.70 | 2.83 |
| 2.58767 | 34.6663 | 327.63 | 2.20 |
| 2.54207 | 35.3083 | 205.97 | 1.38 |
| 2.46002 | 36.5269 | 216.69 | 1.46 |
| 2.40938 | 37.3226 | 255.81 | 1.72 |
| 2.35453 | 38.2254 | 173.61 | 1.17 |
| 2.24550 | 40.1591 | 191.14 | 1.28 |
| 2.22054 | 40.6304 | 94.39 | 0.63 |
| 2.18144 | 41.3918 | 131.06 | 0.88 |
| 2.13303 | 42.3759 | 62.27 | 0.42 |
| 2.09908 | 43.0951 | 177.74 | 1.19 |
| 2.04423 | 44.2732 | 54.00 | 0.36 |

TABLE 3
Polymorph Form C
X-Ray Powder Diffraction Spectral Lines

| d value | 2-θ° | Intensity | Intensity % |
|---|---|---|---|
| 28.98170 | 3.0486 | 2035.93 | 19.64 |
| 27.86354 | 3.1710 | 1630.41 | 15.73 |
| 23.81252 | 3.7106 | 745.81 | 7.19 |
| 18.40502 | 4.8013 | 496.44 | 4.79 |
| 15.27098 | 5.7875 | 10368.10 | 100.00 |
| 11.08304 | 7.9774 | 4318.88 | 41.66 |
| 9.26468 | 9.5465 | 137.91 | 1.33 |
| 8.95231 | 9.8804 | 110.22 | 1.06 |
| 7.82234 | 11.3120 | 476.13 | 4.59 |
| 6.97332 | 12.6947 | 683.66 | 6.59 |
| 5.93875 | 14.9178 | 820.25 | 7.91 |
| 5.52977 | 16.0281 | 576.32 | 5.56 |
| 4.89445 | 18.1100 | 1417.44 | 13.67 |
| 4.87567 | 18.1955 | 1147.66 | 11.07 |
| 4.74590 | 18.6974 | 381.38 | 3.68 |
| 4.51112 | 19.6800 | 453.46 | 4.37 |
| 4.35909 | 20.3735 | 317.56 | 3.06 |
| 3.92030 | 22.6826 | 100.58 | 0.97 |
| 3.71125 | 23.9787 | 126.80 | 1.22 |
| 3.26416 | 27.3226 | 135.73 | 1.31 |
| 2.91505 | 30.6707 | 77.07 | 0.74 |
| 2.76171 | 32.4192 | 41.17 | 0.40 |

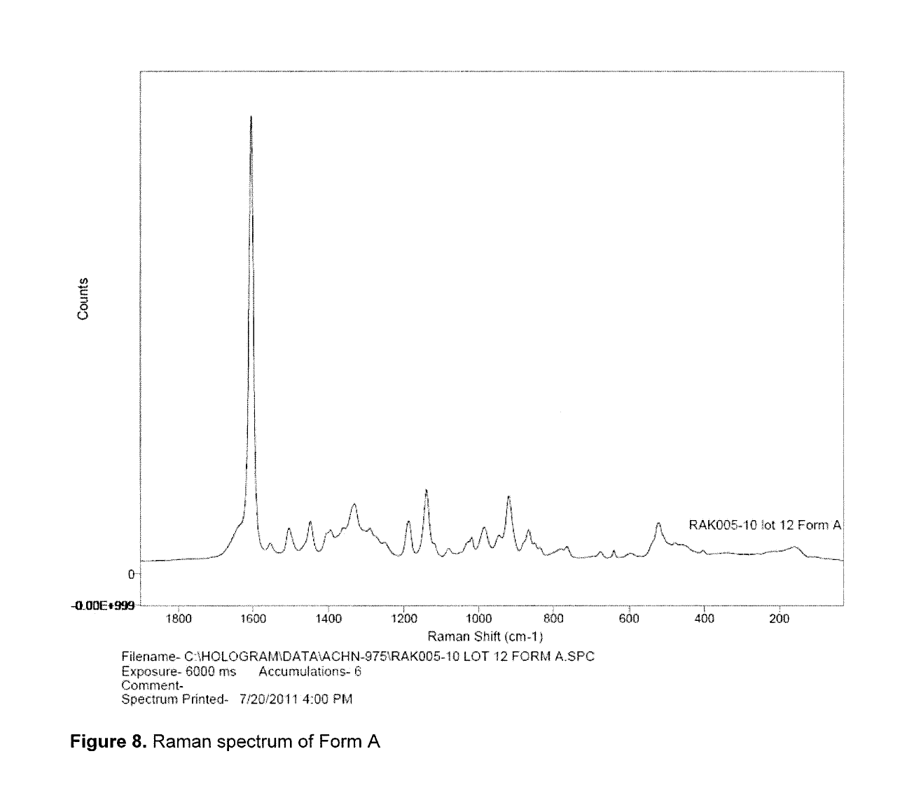

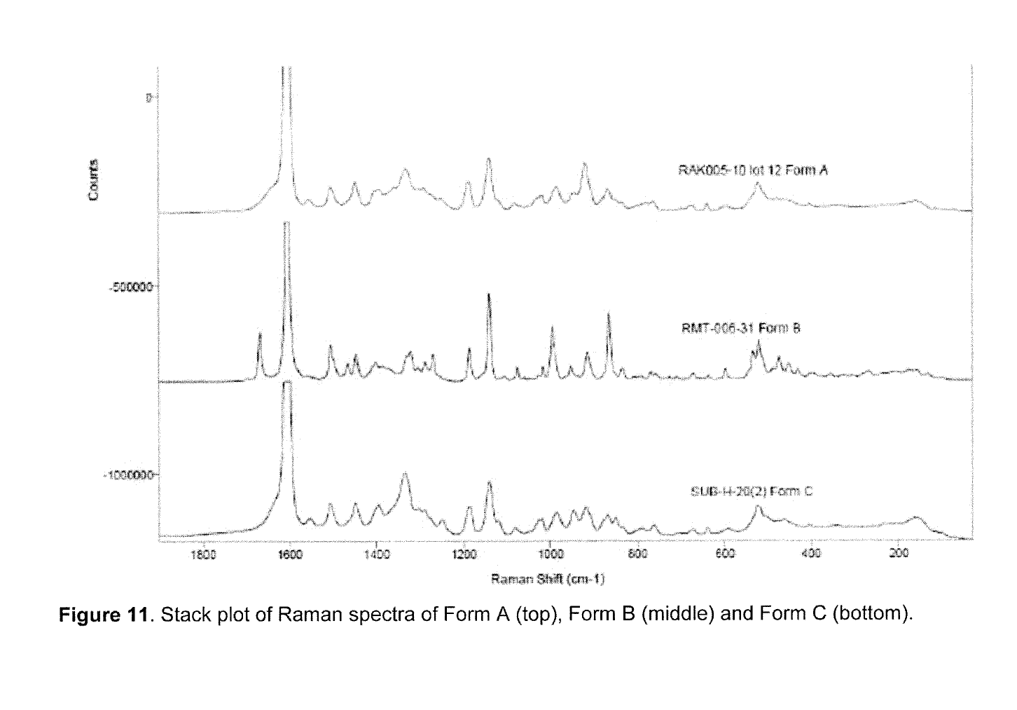

The Raman spectra of polymorph forms A, B and C are presented in FIGS. 8, 9 and 10. In addition, a stack plot of the Raman spectra of polymorph Forms A, B and C is presented in FIG. 11. The Raman spectra was acquired on a Raman accessory module interfaced to a Kaiser RamanRXN1. Analysis was performed using the following conditions:
Raman Source: 785 nm laser
Microscope Objective 1.2 mm
Single Exposure Time: 12 sec or 16 sec
Co-additions: 12
Enabled Exposure options: Cosmic Ray filtering, Dark Subtraction, Intensity Calibration

Polymorph Forms A, B and C may be prepared as set forth in the following examples. Polymorph Forms A, B and C may also be prepared by crystallization from a crystallization solvent containing Compound I. As used herein, the term “crystallization solvent” means a solvent or combination of solvents from which Compound I is preferentially crystallized as polymorph Form A, B or C. Representative crystallization solvents include polar solvents, nonpolar solvents, protic solvents and aprotic solvents, and more specifically include methanol (MeOH), ethanol (EtOH), isopropanol (IPA), tert-amyl alcohol (tAmOH), butanol (nBuOH), tetrahydrofuran (THF), 2-methyl tetrahydrofuran (2-MeTHF), dioxane, acetone, dimethylformamide (DMF), dimethyacetamide (DMA), dimethylsulfoxide (DMSO), N-methyl-2-pyrrolodine (NMP), acetic acid, acetonitrile (MeCN), methyl acetate (MeOAc), ethyl acetate (EtOAc) and methyl ethyl ketone (MEK).

Compound I may be introduced into the crystallization solvent in either a solid or liquid form. When added as a solid, Compound I may be in the form of a solid powder or any other solid form that aids its dissolution within the crystallization solvent. When added as a liquid, Compound I may first be dissolved in a co-solvent to yield a co-solvent solution, which is then combined with the crystallization solvent. The concentration of Compound I within the co-solvent solution may range from 0.1% by weight to the saturation point. This concentration will, of course, vary depending upon the temperature at which the co-solvent solution is held, with warmer temperatures generally allowing for the preparation of more concentrated solutions of Compound I. In general, the co-solvent should aid in the dissolution of Compound I, but not negatively interfere with the formation of polymorph Form A, B or C from the resulting crystallization solvent. Suitable co-solvents include the same solvents as identified above for the crystallization solvent. Further, the co-solvent and the crystallization solvent may be the same or different. For example, both the crystallization solvent and the co-solvent may be acetic acid, or they may be different solvents (or combinations thereof).

In one embodiment, the co-solvent solution containing Compound I is added to the crystallization solvent or, alternatively, the crystallization solvent is added to the co-solvent solution. In still another embodiment, the co-solvent solution may be at or above ambient temperature (e.g., heated), while the temperature of the crystallization solvent may be below (e.g., chilled), above (e.g., heated) or at ambient temperature. Alternatively, the co-solvent solution can undergo a solvent exchange and form a solution or heterogeneous mixture of the crystallization solvent and Compound I. For example, Compound I may be dissolved in a first solvent, followed by addition to a second solvent, and then followed by removal of all or part of the first solvent (e.g., by distillation).

As noted above, polymorph Forms A, B and C have activity as antibacterial agents, particularly in the treatment of infections caused by gram-negative bacteria by inhibiting activity of UDP-3-O—(R-3-hydroxydecanoyl)-N-acetylglucosamine deacetylase (LpxC).

In one aspect, the invention provides a method of inhibiting a deacetylase enzyme in a gram-negative bacteria, thereby affecting bacterial growth, comprising administering to a subject in need of such inhibition a polymorph of Form A, B or C.

In another aspect, the invention provides a method of inhibiting LpxC, thereby modulating the virulence of a bacterial infection, comprising administering to a subject in need of such inhibition a polymorph of Form A, B or C. In certain embodiments of the method of inhibiting LpxC using a polymorph of the present invention, the IC₅₀ value of the polymorph is less than or equal to 10 μM with respect to LpxC. In other embodiments, the IC₅₀ value is less than or equal to 1 M, is less than or equal to 0.1 M, is less than or equal to 0.050 μM, is less than or equal to 0.030 μM, is less than or equal to 0.025 μM, or is less than or equal to 0.010 μM.

In another aspect, the invention provides a method for treating a subject having a gram-negative bacterial infection comprising administering to the subject in need thereof an antibacterially effective amount of a polymorph of Form A, B or C.

In another aspect, the invention provides a method of administering a therapeutically effective amount of a polymorph of Form A, B or C to a subject infected with a fermentative or non-fermentative gram-negative bacteria. Examples of fermentative or non-fermentative gram-negative bacteria include Pseudomonas aeruginosa, Stenotrophomonas maltophila, Burkholderia cepacia, Alcaligenes xylosoxidans, and Enterobacteriaceae, Haemophilus, Franciscellaceae (e.g., Franciscella tularensis) and Neisseria species.

In another aspect, the invention provides a method of administering an inhibitory amount of a polymorph of Form A, B or C to gram-negative bacteria, such as Enterobacteriaceae which is selected from the group consisting of organisms such as Serratia, Proteus, Klebsiella, Enterobacter, Citrobacter, Salmonella, Providencia, Yersinia (e.g., Yersinia pestis), Morganella, Cedecea, Edwardsiella species and Escherichia coli.

In certain embodiments, the subject may be a mammal, and in some embodiments, a human.

Bacterial infections susceptible to treatment according to the present invention include primary infections and co-infections caused by a species of bacteria and one or more additional infectious agents such as, for example, bacteria, virus, parasite and fungus.

Polymorphs of the invention can be used for treating conditions caused by the bacterial production of endotoxin and, in particular, by gram-negative bacteria and bacteria that use LpxC in the biosynthesis of lipopolysaccharide (LPS) or endotoxin.

Polymorphs of the invention also are useful in treating conditions that are caused or exacerbated by the bacterial production of lipid A and LPS or endotoxin, such as sepsis, septic shock, systemic inflammation, localized inflammation, chronic obstructive pulmonary disease (COPD) and acute exacerbations of chronic bronchitis (AECB). For sepsis, septic shock, systemic inflammation, localized inflammation, chronic obstructive pulmonary disease (COPD) and acute exacerbations of chronic bronchitis (AECB), representative non-antibacterial agents include antiendotoxins including endotoxin receptor-binding antibodies, endotoxin-binding antibodies, anti-CD14-binding protein antibodies, antilipopolysaccharide-binding protein antibodies and tyrosine kinase inhibitors.

In treatment of serious or chronic respiratory tract infections, polymorphs of the present invention may also be used with non-antibacterial agents administered via inhalation. Representative non-antibacterial agents used in this treatment include anti-inflammatory steroids, non-steroidal anti-inflammatory agents, bronchiodilators, mucolytics, anti-asthma therapeutics and lung fluid surfactants. In particular, the non-antibacterial agent may be albuterol, salbuterol, budesonide, beclomethasone, dexamethasone, nedocromil, beclomethasone, fluticasone, flunisolide, triamcinolone, ibuprofin, rofecoxib, naproxen, celecoxib, nedocromil, ipratropium, metaproterenol, pirbuterol, salmeterol, formoterol, indacaterol, bronchiodilators, mucolytics, calfactant, beractant, poractant alfa, surfaxin or pulmozyme (also called domase alfa).

Polymorphs of the invention can be used alone or in combination with a second antibacterial agent for the treatment of a serious or chronic respiratory tract infection including serious lung and nosocomial infections such as those caused by Enterobacter aerogenes, Enterobacter cloacae, Escherichia coli, Klebsiella pneumoniae, Klebsiella oxytoca, Proteus mirabilis, Serratia marcescens, Stenotrophomonas maltophilia, Pseudomonas aeruginosa, Burkholderia cepacia, Alcaligenes xylosoxidans, Flavobacterium meningosepticum, Providencia stuartii and Citrobacterfreundi, community lung infections such as those caused by Haemophilus Influenzae, Legionella species, Moraxella catarrhalis, Branhamella catarrhalis, Enterobacter species, Klebsiella species, and Proteus species, infections caused by other bacterial species such as Neisseria species, Shigella species, Salmonella species, Helicobacter pylori, Vibrionaceae and Bordetella species, as well as infections caused by a Brucella species, Francisella tularensis and/or Yersinia Pestis.

Pharmaceutical compositions of the present invention comprise a therapeutically effective amount of a polymorph of Form A, B or C, formulated together with one or more pharmaceutically acceptable carriers or diluents. As used herein, the term “pharmaceutically acceptable carrier” means a non-toxic, inert solid, semi-solid or liquid filler, diluent, encapsulating material or formulation auxiliary of any type. Some examples of materials that can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; alginic acid; pyrogen-free water, isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. The pharmaceutical compositions of this invention can be administered to humans and other animals orally, rectally, parenterally (as by intravenous, intramuscular or subcutaneous injection), intracisternally, intravaginally, intraperitoneally, topically (as by powders, ointments, or drops), bucally, or as an oral or nasal spray, or a liquid aerosol or dry powder formulation for inhalation.

Liquid dosage forms for oral administration include pharmaceutically acceptable emulsions, microemulsions, solutions, suspensions, syrups and elixirs. In addition to the active polymorphs, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, 1% lidocaine, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions that can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution that, in turn, may depend upon crystal size and crystalline form. Alternatively, delayed absorption of a parenterally administered drug form may be accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations may also be prepared by entrapping the drug in liposomes or microemulsions that are compatible with body tissues.

Compositions for rectal or vaginal administration are preferably suppositories that can be prepared by mixing the polymorphs of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active polymorph is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, acetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Solid compositions of a similar type may also be employed as fillers in soft and hard-filled gelatin capsules using such excipients as lactose or milk sugar as well as high molecular weight polyethylene glycols and the like.

The antibacterial polymorphs can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active polymorph may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a polymorph of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulations, ear drops, and the like are also contemplated as being within the scope of this invention.

The ointments, pastes, creams and gels may contain, in addition to an active polymorph of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Polymorphs of the invention may also be formulated for delivery as a liquid aerosol or inhalable dry powder. Liquid aerosol formulations may be nebulized predominantly into particle sizes that can be delivered to the terminal and respiratory bronchioles where bacteria reside in subjects with bronchial infections, such as chronic bronchitis and pneumonia. Pathogenic bacteria are commonly present throughout airways down to bronchi, bronchioli and lung parenchema, particularly in terminal and respiratory bronchioles. During exacerbation of infection, bacteria can also be present in alveoli. Liquid aerosol and inhalable dry powder formulations are preferably delivered throughout the endobronchial tree to the terminal bronchioles and eventually to the parenchymal tissue.

Aerosolized formulations of the invention may be delivered using an aerosol forming device, such as a jet, vibrating porous plate or ultrasonic nebulizer, preferably selected to allow the formation of a aerosol particles having with a mass medium average diameter predominantly between 1 to 5 μm. Further, the formulation preferably has balanced osmolarity ionic strength and chloride concentration, and the smallest aerosolizable volume able to deliver effective dose of the polymorphs of the invention to the site of the infection. Additionally, the aerosolized formulation preferably does not impair negatively the functionality of the airways and does not cause undesirable side effects.

Aerosolization devices suitable for administration of aerosol formulations of the invention include, for example, jet, vibrating porous plate, ultrasonic nebulizers and energized dry powder inhalers, that are able to nebulize the formulation of the invention into aerosol particle size predominantly in the size range from 1-5 pm. Predominantly in this application means that at least 70% but preferably more than 90% of all generated aerosol particles are 1 to 5 μm range. A jet nebulizer works by air pressure to break a liquid solution into aerosol droplets. Vibrating porous plate nebulizers work by using a sonic vacuum produced by a rapidly vibrating porous plate to extrude a solvent droplet through a porous plate. An ultrasonic nebulizer works by a piezoelectric crystal that shears a liquid into small aerosol droplets. A variety of suitable devices are available, including, for example, AeroNeb and AeroDose vibrating porous plate nebulizers (AeroGen, Inc., Sunnyvale, Calif.), Sidestream7 nebulizers (Medic-Aid Ltd., West Sussex, England), Pari LC7 and Pari LC Star7 jet nebulizers (Pari Respiratory Equipment, Inc., Richmond, Va.), and Aerosonic (DeVilbiss Medizinische Produkte (Deutschland) GmbH, Heiden, Germany) and UltraAire7 (Omron Healthcare, Inc., Vernon Hills, Ill.) ultrasonic nebulizers.

Polymorphs of the invention may also be formulated for use as topical powders and sprays that can contain, in addition to the polymorphs of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a polymorph to the body. Such dosage forms can be made by dissolving or dispensing the polymorph in the proper medium. Absorption enhancers can also be used to increase the flux of the polymorph across the skin. The rate can be controlled by either providing a rate controlling membrane or by dispersing the polymorph in a polymer matrix or gel.

According to the methods of treatment of the present invention, bacterial infections are treated or prevented in a subject such as a human or lower mammal by administering to the subject a therapeutically effective amount of a polymorph of Form A, B or C, in such amounts and for such time as is necessary to achieve the desired result. By a “therapeutically effective amount” of a polymorph of the invention is meant a sufficient amount of the polymorph to treat bacterial infections, at a reasonable benefit/risk ratio applicable to any medical treatment. It will be understood, however, that the total daily usage of the polymorphs of the present invention will be decided by the attending physician within the scope of sound medical judgment. The specific therapeutically effective dose level for any particular subject will depend upon a variety of factors including the disorder being treated and the severity of the disorder; the activity of the specific polymorph employed; the specific composition employed; the age, body weight, general health, sex and diet of the subject; the time of administration, route of administration, and rate of excretion of the specific polymorph employed; the duration of the treatment; drugs used in combination or coincidental with the specific compound employed; and like factors well known in the medical arts.

The total daily dose of the polymorphs of this invention administered to a human or other mammal in single or in divided doses can be in amounts, for example, from 0.01 to 200 mg/kg body weight or more usually from 0.1 to 50 mg/kg body weight. In certain embodiments, the total daily dose administered to a human or other mammal is from 1.0 to 100 mg/kg body weight or from 5.0 to 25 mg/kg body weight. Single dose compositions may contain such amounts or submultiples thereof to make up the daily dose. In general, treatment regimens according to the present invention comprise administration to a subject in need of such treatment from about 10 mg to about 15 g of a polymorph of this invention per day in single or multiple doses, more usually, from 100 mg to 5 g, and even more usually from 250 mg to 1 g per day in single or multiple doses.

Methods of formulation are well known in the art and are disclosed, for example, in Remington: The Science and Practice of Pharmacy, Mack Publishing Company, Easton, Pa., 19th Edition (1995). Pharmaceutical compositions for use in the present invention can be in the form of sterile, non-pyrogenic liquid solutions or suspensions, coated capsules, suppositories, lyophilized powders, transdermal patches or other forms known in the art.

A “kit” as used in the instant application includes a container for containing the pharmaceutical compositions and may also include divided containers such as a divided bottle or a divided foil packet. The container can be in any conventional shape or form as known in the art that is made of a pharmaceutically acceptable material, for example a paper or cardboard box, a glass or plastic bottle or jar, a resealable bag (for example, to hold a “refill” of tablets for placement into a different container), or a blister pack with individual doses for pressing out of the pack according to a therapeutic schedule. The container employed can depend on the exact dosage form involved, for example a conventional cardboard box would not generally be used to hold a liquid suspension. It is feasible that more than one container can be used together in a single package to market a single dosage form. For example, tablets may be contained in a bottle that is in turn contained within a box.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process, recesses are formed in the plastic foil. The recesses have the size and shape of individual tablets or capsules to be packed or may have the size and shape to accommodate multiple tablets and/or capsules to be packed. Next, the tablets or capsules are placed in the recesses accordingly and the sheet of relatively stiff material is sealed against the plastic foil at the face of the foil that is opposite from the direction in which the recesses were formed. As a result, the tablets or capsules are individually sealed or collectively sealed, as desired, in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a written memory aid, where the written memory aid is of the type containing information and/or instructions for the physician, pharmacist or other health care provider, or subject, e.g., in the form of numbers next to the tablets or capsules whereby the numbers correspond with the days of the regimen that the tablets or capsules so specified should be ingested or a card that contains the same type of information. Another example of such a memory aid is a calendar printed on the card e.g., as follows “First Week, Monday, Tuesday,” . . . etc. . . . “Second Week, Monday, Tuesday, . . . ” etc. Other variations of memory aids will be readily apparent. A “daily dose” can be a single tablet or capsule or several tablets or capsules to be taken on a given day. When the kit contains separate compositions, a daily dose of one or more compositions of the kit can consist of one tablet or capsule while a daily dose of another one or more compositions of the kit can consist of several tablets or capsules.

Another specific embodiment of a kit is a dispenser designed to dispense the daily doses one at a time in the order of their intended use. Preferably, the dispenser is equipped with a memory-aid, so as to further facilitate compliance with the regimen. An example of such a memory-aid is a mechanical counter that indicates the number of daily doses that has been dispensed. Another example of such a memory-aid is a battery-powered micro-chip memory coupled with a liquid crystal readout, or audible reminder signal that, for example, reads out the date that the last daily dose has been taken and/or reminds one when the next dose is to be taken.

The kits of the present invention may also include, in addition to a polymorph of the present invention, one or more additional pharmaceutically active compounds. For example, the additional compound may be a second antibacterial agent. The additional compounds may be administered in the same dosage form as the polymorph of the present invention or in a different dosage form. Likewise, the additional compounds can be administered at the same time as the polymorph of the present invention or at different times.

The following examples are offered by way of illustration, not limitation.

### EXAMPLES

### Example 1

### Synthesis of Polymorph Form A of N—((S)-3-amino-1-(hydroxyamino)-3-methyl-1-oxobutan-2-yl)-4-(((1R,2R)-2-(hydroxymethyl)cyclopropyl)buta-1,3-diynyl)benzamide (Compound I)

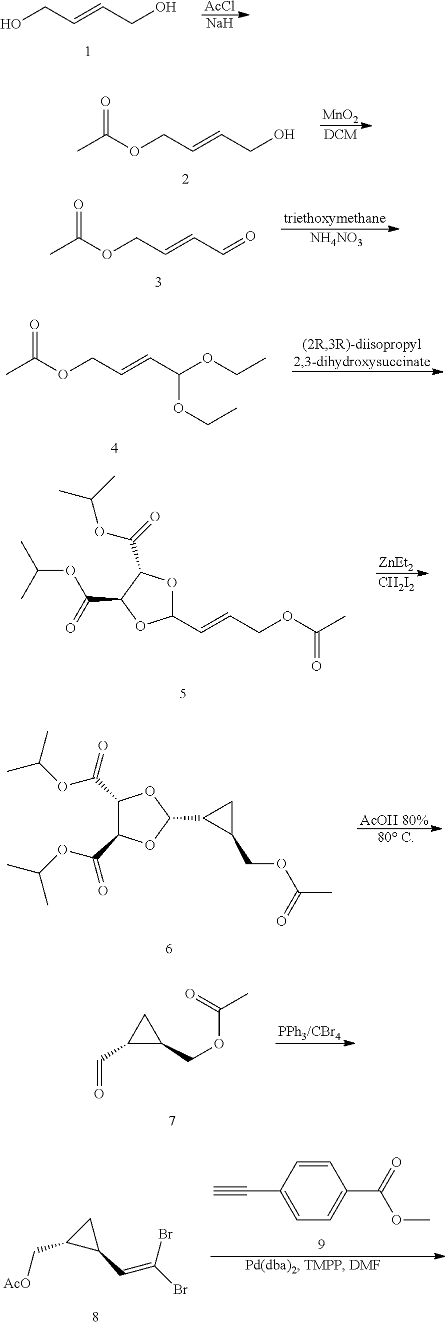

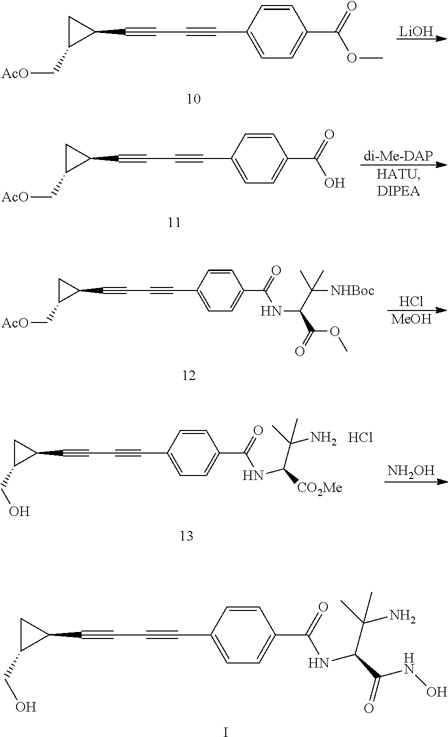

### (E)-4-hydrarxybut-2-enyl acetate (2)

To a solution of (E)-but-2-ene-1,4-diol 1 (264 g, 3.0 mol) at −20° C. in THF (1.5 L) was added sodium hydride (120 g, 3.0 mol) in portions. Upon the addition, the mixture was kept stirring at −20° C. for 30 mins. Then acetyl chloride (235.5 g, 3 mol) was added dropwise, the mixture allowed to warm to rt and kept stirring at rt for another 3 hours. The mixture was filtered and the residue was washed with THF. The combined organic layer were dried and concentrated to give crude 2 which was purified by silica gel column (PE:EA=5:1-2:1) to give 2 (210 g) as a colorless oil. Yield: 54%. ¹HNMR:CP-0005065-043 (CDCl₃, 400 MHz) δ: 5.85 (m, 1H), 5.62 (m, 1H), 4.67 (t, J=6.2 Hz, 2H), 4.26 (t, J=6.0 Hz, 2H), 2.10 (s, 1H), 2.06 (s, 3H).

### (E)-4-oxobut-2-enyl acetate (3)

To a suspension of manganese dioxide (active, 1305 g, 15 mol) in 2.5 L dichloromethane, added (E)-4-hydroxybut-2-enyl acetate 2 (195 g) in portions. The mixture was kept stirring at rt for 48 hours. The mixture was filtered and the residue was washed with dichloromethane. The combined organic layer were dried and concentrated to give crude 3 which was purified by silica gel column (PE:EA=10:1-5:1) to give 3, 130 g as a colorless oil. Yield: 64%. 1 HNMR:CP-0005065-044 (CDCl3,400 M HZ) δ: 10.01 (d, J=6.4 Hz, 1H), 6.52 (m, 1H), 6.10 (m, 1H), 5.08 (m, 2H), 2.10 (s, 3H).

### (E)-4,4-diethoxybut-2-enyl acetate (4)

To a solution of 3 (96.0 g, 0.75 mol) and triethoxymethane (133.2 g, 0.9 mol) in 500 mL ethanol, added ammonium nitrate (3.0 g, 0.038 mol), the mixture was kept stirring at rt for 15 hours. The mixture was diluted with 800 mL EtOAc and washed with saturated sodium bicarbonate. The aqueous layer was back extracted with EtOAc (300 mL×2). The combined organic layers were dried and concentrated to give crude 4 (140 g) as a red oil which was used for the next step without for further purification.

### (4R,5R,E)-disopropyl-2-(3-acetoxyprop-1-enyl)-1,3-dioxolane-4,5-dicarboxylate (5)

To a solution of 4 (60.6 g, 0.3 mol) and (2R,3R)-diisopropyl 2,3-dihydroxysuccinate (77.2 g, 0.33 mol) in 500 ml benzene, added PPTS (3.8 g, 15 mmol), the mixture was heated to 90° C. to distill off the ethanol for 15 hours. The mixture was cooled to rt and concentrated in vacuum. Purified by silica gel with (PE:EA=50:1-30:1), 38.5 g 5 as a colorless oil was obtained. Yield: 37.3%. GCMS:CP-0005065-070-2 (85% pure).

### (4R,5R)-diisopropyi-2-((1S,2R)-2-(acetoymethyl)cycopropyl)-1,3-dioxolane-4,5-dicarboxylate (6)

To a solution of 5 (34.4 g, 0.1 mol) in hexane (1.5 L), added diethyl zinc (IM in hexane, 500 mL) in portion under argon at −20° C. Upon the addition, diiodoethane (267.8 g, 1 mol) was added dropwise below −20° C. with strong stirring. The mixture allowed warm to rt and kept stirring for another 8 hours. The reaction mixture was quenched by 800 mL cold aqueous ammonium chloride, then extracted with ether (800 mL×5). The combined organic layers were washed with aqueous sodium thiosulfate, water, brine, then dried and concentrated to give crude 6. The residue was purified by silica gel column chromatography (PE:EA=30:1-10:1) to provide 6 (16 g) as a colorless oil. Yield: 44.7%. ¹HNMR:PO5 (CDCl3,400 MHZ) δ: 5.13 (m, 2H), 4.95 (d, J=5.6 Hz, 1H), 4.67 (d, J=3.6 Hz, 1H), 4.57 (d, J=4.0 Hz, 1H), 4.07 (m, 1H), 3.88 (m, 1H), 2.06 (s, 3H), 1.38 (s, 12H), 1.23 (m, 1H), 0.83 (m, 1H), 0.66 (m, 1H).

### (1R,2R)-2-formylcyclopropyl)methyl acetate (7)

A mixture of 6 (14.3 g, 40 mmol) in 140 mL 80% acetic acid was heated to 80° C. and kept stirring at this temperature for 2 hours. When TLC showed little 6 remaining, the mixture was added to 300 mL saturated sodium bicarbonate dropwise, then extracted with dichloromethane (200 mL×3). The combined organic layers were washed with water, brine dried and concentrated to give crude 7 which was purified by silica gel column (PE:EA=10:1-5:1) to give 7, 3.5 g as a colorless oil. Yield: 62%. 1H NMR:CP-0005065-072 (CDCl3,400 M HZ) δ: 9.15 (s, 1H), 4.11 (m, 1H), 3.91 (m, 1H), 2.08 (s, 3H), 2.10 (s, 3H), 1.88 (m, 2H), 1.39 (m, 1H), 1.12 (m, 1H).

### (1R,2R)-2-(2,2-dibromovinyl)cyclopropyl)methyl acetate (8)

To a solution of carbon tetrabromide (13.9 g, 42 mmol) in dichloromethane 30 mL, added a solution of triphenylphosphine (22.0 g, 84 mmol) in 50 mL dichloromethane dropwise at −20° C. under argon. The mixture was kept stirring at this temperature for half an hour, then cooled to −78° C. Added a solution of 7 (3.00 g, 21 mmol) in 40 mL dichloromethane dropwise, maintained the temperature for another half an hour. The mixture was allowed warm to ambient temperature over 30 min. The solvent was removed and the resulting residue was purified by silica gel column chromatography (PE:EA=100:1-50:1) to give 8 as a colorless oil 4.3 g. Yield: 69%. ¹HNMR:CP-0005065-075 (CDCl₃, 400 MHz) δ: 5.85 (d, J=8.8 Hz, 1H), 3.98 (m, 2H), 2.09 (m, 3H), 1.61 (m, 1H), 1.35 (m, 1H), 0.88 (m, 2H).

### Methyl 4-(((1R,2R)-2-(acetoxymethyl)cyclopropyl)buta-1,3-diynyl)benzoate (10)

To a solution of 8 (4.3 g, 14.5 mmol), Pd₂dba₂ (86.3 mg, 0.15 mmol), tri(4-methylphenyl)phosphine (204.2 mg, 0.58 mmol), triethylamine (4.35 g, 43.5 mmol) in DMF 100 mL, treated with methyl 4-ethynylbenzoate 9 (2.56 g, 16 mmol) under argon. The mixture was kept stirring at rt for 5 hours. When TLC showed little compound 8 remaining, the reaction was diluted with EtOAc (300 mL) and washed with water (3×100 mL), the organic layer was dried and concentrated to give crude 10 which was purified by silica gel column chromatography PE:EA=50:1-30:1 to give 10 2.0 g as a yellow solid. Yield: 46.5%, LCMS:CP-0005065-085-2 (ESI) m/z=297 (M+1) purity: 92.4% (214 nm).

### 4-(((1R,2R)-2-(hydroxymethyl)cyclopropyl)buta-1,3-diynyl)benzoic acid (11)

10 (1.92 g, 6.5 mmol) was dissolved in THF (40 mL), then added to a solution of sodium hydroxide (2.60 g, 65 mmol) in 10 mL water. The mixture was kept stirring at ambient temperature for 8 hours. When LCMS showed little compound 10 remaining, the solvent was removed under reduced pressure, the residue was diluted with water (50 mL), adjusted the pH to 4.0, extracted with ethyl acetate (4×50 mL), the organic layer was dried and concentrated to give crude 11 1.4 g as a yellow solid which was used for next step without further purification. LCMS:CP-0005065-088-3 (ESI) m/z=241 (M+1) purity: 89% (214 nm). Yield: 89%.

### (S)-methyl-2-(4-(((1R,2R)-2-(acetoxymethyl)cyclopropyl)buta-1,3-diynyl)benzamido)-3-(tert-butoxycarbonylamino)-3-methylbutanoate (12)

To a solution of 11 (1.20 g, 5.0 mmol), HATU (2.34 g, 6 mmol) in DMF 50 mL, treated with (S)-methyl-2-amino-3-(tert-butoxycarbonylamino)-3-methylbutanoate (1.48 g, 6.0 mmoL) and DIPEA (3.58 g, 20 mmoL). The mixture was stirred at ambient temperature for 5 hours. When LCMS showed little compound 11 remaining, the reaction was diluted with EtOAc (100 mL), washed with 5% lithium chloride (3×50 mL), the organic layer was dried and concentrated to give 12 as a yellow oil. Purification by silica gel column chromatography PE:EA=2:1 gave 2.0 g 12 as a colorless oil, yield: 70%, LCMS:CP-0005065-091-3 (ESI) m/z=469 (M+1) purity: 95% (214 nm).

### (S)-methyl-3-amino-2-(4-(((1R,2R)-2-(hydroxymethyl)cyclopropyl)buta-1,3-diynyl)benzamido)-3-methylbutanoate hydrochloride (13)

12 (1.87 g, 4.0 mmol) was dissolved in methanol (50 mL), treated with dry HCl, for 10 min. When LCMS showed little compound 12 remaining, HCl flow was stopped HCl. The solvent was removed under reduced pressure to provide 13 (1.45 g) as a yellow solid. Yield: 91%, LCMS: CP-0005065-096-4-LCMSA019 (ESI) m/z=369 (M+l) purity: 98% (214 nm). ¹H NMR:CP-0005065-096-4 (DMSO-d₆, 400 MHz) 8:9.04 (d, J=6.8 Hz, 1H), 8.36 (s, 3H), 7.98 (d, J=6.4 Hz, 2H), 7.64 (d, J=6.8 Hz, 2H), 4.89 (d, J=6.8 Hz, 1H), 3.73 (s, 3H), 3.44 (m, 1H), 3.22 (m, 1H), 1.48 (m, 2H), 1.40 (s, 6H), 0.94 (m, 2H).

### Polymorph Form A of N—((S)-3-amino-1-(hydroxyamino)-3-methyl-1-oxobutan-2-yl)-4-(((1R,2R)-2-(hydroxymethyl)cyclopropyl)buta-1,3-diynyl)benzamide (I)

(S)-methyl-3-amino-2-(4-(((I R,2R)-2-(hydroxymethyl)cyclopropyl) buta-1,3-diynyl)benzamido)-3-methylbutanoate hydrochloride 13 (29.2 g, 72 mmol) was taken up in isopropanol (120 mL) and tetrahydrofuran (18 mL) and cooled in an ice bath. 50% aqueous hydroxylamine (105 mL, 1.58 mol, 22 equiv) was added and the mixture allowed to react at 0-5° C. until predominantly complete by HPLC. Solvents were removed at 0° C. under vacuum and acetic acid (250 mL) was added slowly. After the addition of water (30 mL) and acetonitrile (10 mL), the mixture was filtered and purified by reverse-phase HPLC (gradient of 2-45% acetonitrile in water, each containing 0.1% acetic acid, over 125 minutes) and the desired fractions pooled and lyophilized to give N—((S)-3-amino-1-(hydroxyamino)-3-methyl-1-oxobutan-2-yl)-4-(((1R,2R)-2-(hydroxymethyl)cyclopropylcyclopropy)buta-1,3-diynyl)benzamide I as its acetate salt (white solid, 16.6 g). To isolate the freebase of I, this material was dissolved in water (200 mL) and 2-methyltetrahydrofuran (10 mL). A solution of 0.3 M sodium carbonate (80 mL) was added to pH 8-9. This aqueous solution was extracted three 5 times with 225 mL each 2-methyltetrahydrofuran, and twice with 100 mL each tetrahydrofuran. The five organic layers were combined and dried twice over sodium sulfate. The solution was concentrated under vacuum to provide a white solid. This solid was triturated with ethyl acetate (150 mL), isolated by filtration, and dried under vacuum to provide I as its freebase (11.8 g, 32.2 mmol, 44% yield). Solids obtained from this procedure have been characterized by DSC (FIG. 1), XRPD (FIG. 4) and Raman spectroscopy (FIG. 8) to determine the crystalline form. Mass spec data: expected (M+1): 370.4, observed: 370.2. ¹H NMR (400 MHz, DMSO-d₆): Proton nmr (400 MHz, dmso-d6): 8.1 (br s, 1H), 7.86 (d, 2H, J=8.0), 7.61 (d, 2H, J=8.0), 4.71 (br. S, 1H) 4.29 (s, 1H) 3.43 (dd, 1H, J=4.8, 10.8 Hz), 3.26 (1H, J=5.2, 11.4 Hz), 1.42-1.49 (m, 2H), 1.09 (s, 3H), 1.01 (s, 3H), 0.96-0.89 (m, 2H).

### Example 2

### Preparation of Polymorph Form B

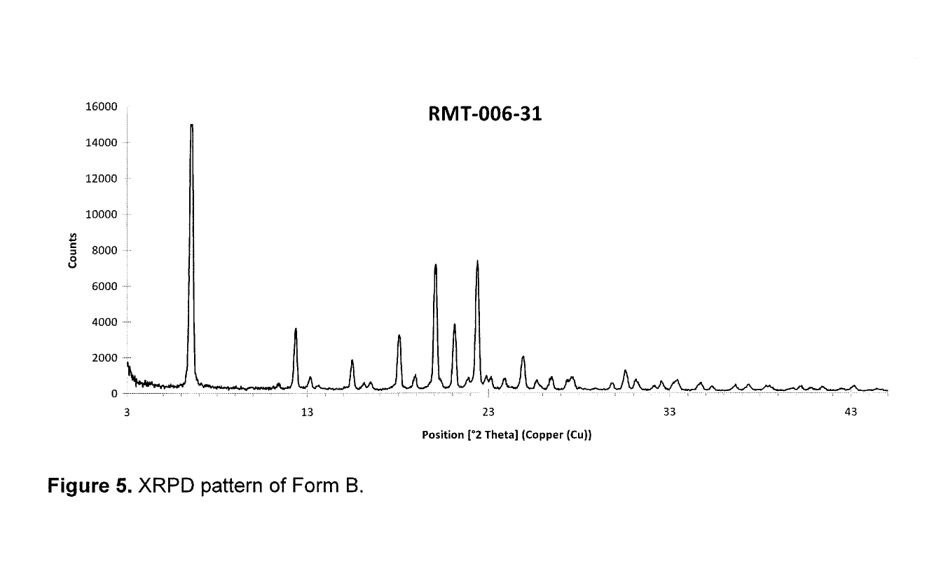

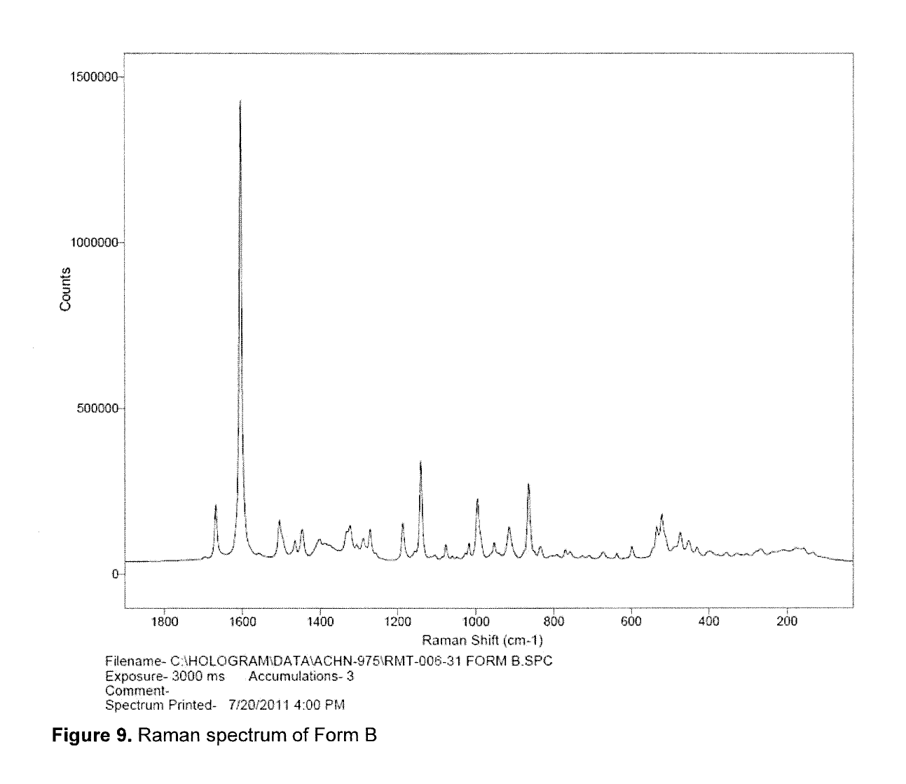

20 mL of a solution of 20% isopropanol in water was prepared in a 50 mL flask equipped with stir bar. To this was added 2 g of I (Example 1) as a solid in one portion. The resulting white slurry was allowed to stir for 24 h at 20-25° C., then isolated by filtration. The solids were washed with 10 mL of a solution of 20% isopropanol in water, and dried in a vacuum oven overnight. 1.65 g (83% yield) of white solids were isolated and analyzed by HPLC for purity. The solids were further characterized by DSC (FIG. 2), XRPD (FIG. 5) and Raman spectroscopy (FIG. 9) to determine the crystalline form.

### Example 3

### Preparation of Polymorph Form C

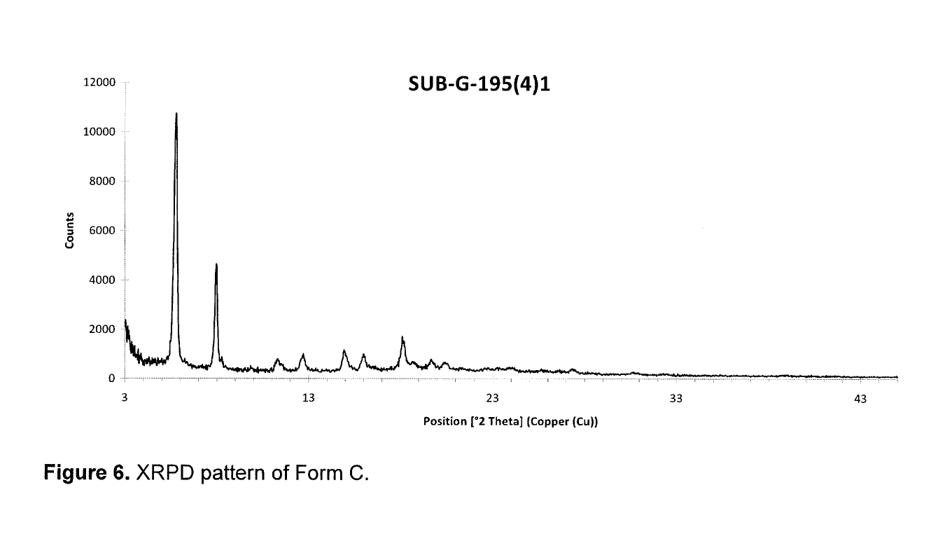

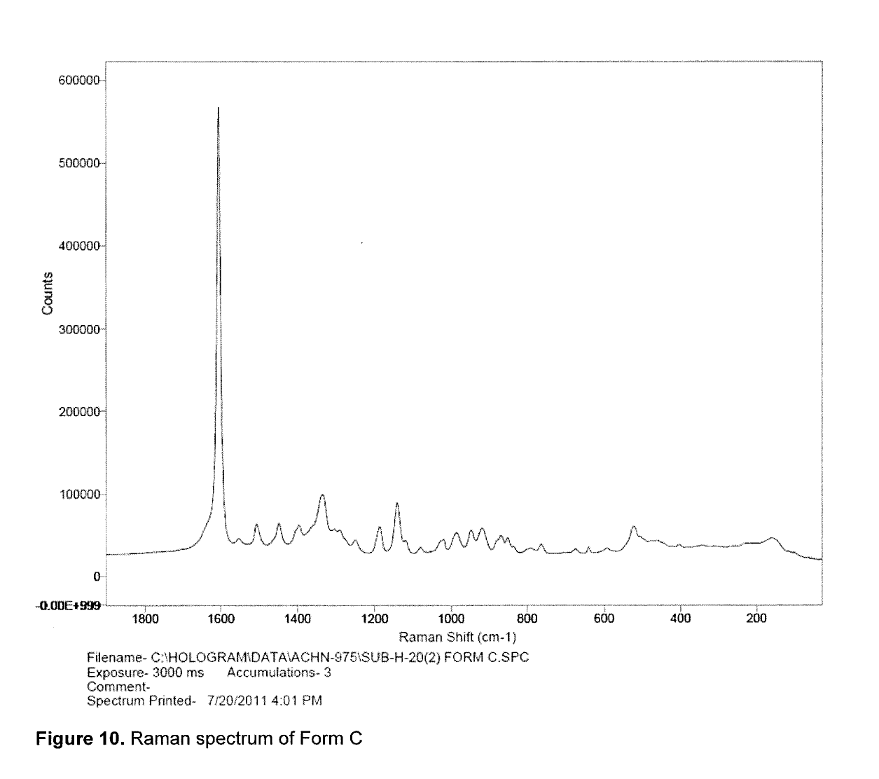

Approximately 250 mg of I (Example 1) was weighed into a 20-mL vial equipped with a magnetic stir bar. 5.0 mL nBuOH was added to ensure dissolution at 50° C. The solution was polished filtered through a 0.45 micron syringe filter into a preheated 20-mL vial. The vial was cooled to room temperature with magnetic stirring at 20° C./hr. Solids were recovered by filtration and dried overnight at room temperature under vacuum. The dried solids were analyzed by DSC (FIG. 3), XRPD (FIG. 6) and Raman spectroscopy (FIG. 10) to determine the crystalline form.

### Example 4

### Stability at Elevated Humidity

Approximately 30 mg of Form A, Form B and Form C were placed on an XRPD slide and kept inside a glass jar containing a water solution saturated with NaCl. The solution maintains 75% relative humidity inside the jar. The container was covered with aluminum foil to prevent exposure to light. The sample was kept in this environment for seven days at room temperature. After this time period the slide was removed from the chamber and analyzed immediately by XRPD (see Table 4).

TABLE 4

| XRPD Initial Form | XRPD Final Form |
|---|---|
| A | A |
| B | B |
| C | C |

### Example 5

### Stability at Elevated Temperature

Approximately 10 mg of Form A, Form B and Form C were weighed to individual 4-mL amber vials covered with a kimwipe and stored in an oven at 60° C. and ambient pressure. After seven days of exposure, the solids were be analyzed by XRPD to check for form conversion and HPLC for purity (see Table 5).

TABLE 5

| | | | | % AUC |
|---|---|---|---|---|
| Initial Amt | XRPD Initial | XRPD Final | % AUC | (Following |
| (mg) | Form | Form | (Control) | exposure) |
| 12.3 | A | A | 99.2 | 94.5 |
| 11.8 | B | B | 99.6 | 97.4 |
| 13.6 | C | C | 99.0 | 95.5 |

### Example 6

### Single Form Slurry Experiments

Approximately 30 mg of Form A, Form B and Form C were weighed into 2-mL HPLC clear vials equipped with magnetic stir bars. 0.5 mL of solvent was added to achieve a free-flowing slurry and allowed to equilibrate at room temperature. Samples were protected from light by covering the vials with aluminum foil. All slurries were isolated via centrifuge filtration after 1 day and 1 week of equilibration. The solids were dried overnight under vacuum at room temperature and analyzed by XRPD (see Table 6). In the case of Form A and Form C it was difficult to achieve a free flowing slurry due to adherence of the stir bar to the bottom of the vial. The vial was sonicated and vortexed a few times to free up the stir bar.

TABLE 6

| Initial | | | | XRPD Final |
|---|---|---|---|---|
| Amt | | XRPD Initial | XRPD Final | Form |
| (mg) | Solvent System | Form (Amt) | Form (1 day) | (7 days) |
| 26.4 | IPA:H₂O (20:80) | A | B | B |
| 27.6 | IPA:IBAc (10:90) | A | A | A |
| 29.7 | IPA:H₂O (20:80) | B | B | B |
| 27.6 | IPA:IBAc (10:90) | B | B | B |
| 30.3 | IPA:H₂O (20:80) | C | B | B |
| 27.3 | IPA:IBAc (10:90) | C | A | A |

* IBAc = Isobutyl acetate

### Example 7

### Competitive Slurry Experiments

Approximately 10 mg of Form A, B and C were weighed into 2.0-mL vials equipped with a stir bar. 0.5 mL of solvent was added and the resulting free flowing slurry was allowed to equilibrate with stirring at room temperature. Samples were pulled at one day and seven day time points, filtered through 0.45 micron centrifuge filters, dried overnight at room temperature under vacuum and analyzed by XRPD (see Table 7).

TABLE 7

| Initial | | | | XRPD Final |
|---|---|---|---|---|
| Amt | | XRPD Initial | XRPD Final | Form |
| (mg) | Solvent System | Form | Form (1 day) | (7 days) |
| 12.5 | IPA:H₂O (20:80) | A | B | B |
| 13.8 | | B | | |
| 10.2 | | C | | |
| 10.3 | IPA:IBAc (10:90) | A | A + B | B |
| 10.8 | | B | | |
| 11.6 | | C | | |

* IBAc = Isobutyl acetate

### Example 8

### Antimicrobial Activity

### Bacterial Screens and Cultures

Bacterial isolates were cultivated from −70° C. frozen stocks by overnight passages at 35° C. in ambient air on Mueller-Hinton agar (Beckton Dickinson, Franklin Lakes, N.J.). Clinical isolates tested were obtained from various geographically diverse hospitals in the US and abroad (Focus Diagnostics, Herndon, Va. and JMI, North Liberty, Iowa). Quality control strains were from the American Type Culture Collection (ATCC; Rockville, Md.).

### Susceptibility Testing

Minimum Inhibitory Concentrations (MICs) were determined by the broth microdilution method in accordance with the Clinical and Laboratory Standards Institute (CLSI) guidelines. In brief, organism suspensions were adjusted to a 0.5 McFarland standard to yield a final inoculum between 3×10⁵ and 7×10⁵ colony-forming units (CFU)/mL. Drug dilutions and inocula were made in sterile, cation adjusted Mueller-Hinton Broth (Beckton Dickinson). An inoculum volume of 100 μL was added to wells containing 100 μL of broth with 2-fold serial dilutions of drug. All inoculated microdilution trays were incubated in ambient air at 35° C. for 18-24 h. Following incubation, the lowest concentration of the drug that prevented visible growth (OD600 nm<0.05) was recorded as the MIC. Performance of the assay was monitored by the use of laboratory quality-control strains and levofloxacin, a compound with a defined MIC spectrum, in accordance with CLSI guidelines. Data for N—((S)-3-amino-1-(hydroxyamino)-3-methyl-1-oxobutan-2-yl)-4-(((1R,2R)-2-(hydroxymethyl)cyclopropyl)buta-1,3-diynyl)benzamide trifluoroacetate salt and freebase are shown in Table 8 below.

TABLE 8
Minimum Inhibitory Concentrations (MICs)

| Cmp# | APAE001 | AECO001 | APAE002 | AKPN001 |
|---|---|---|---|---|
| I | A | A | A | A |
| (trifluoroacetate salt) | | | | |
| I | A | A | A | A |
| (freebase) | | | | |

### MIC Key:

A=MIC of 2.0 μg/mL or less

B=MIC of greater than 2.0 g/mL to 16.0 g/mL

C=MIC of greater than 16.0 μg/mL

AECO001 is E. coli ATCC25922; APAE001 is Pseudomonas aeruginosa ATCC27853; AKPN001 is Klebsiella pneumoniae ATCC43816; APAE002 is a clinical isolate of Pseudomonas aeruginosa expressing a normal level of efflux activity.

While particular embodiments of the present invention have been shown and described herein for purposes of illustration, it will be understood, of course, that the invention is not limited thereto since modifications may be made by persons skilled in the art, particularly in light of the foregoing teachings, without deviating from the spirit and scope of the invention. Accordingly, the invention is not limited except as by the appended claims.

All of the U.S. patents, U.S. patent application publications, U.S. patent applications, foreign patents, foreign patent applications and non-patent publications referred to in this specification are incorporated herein by reference, in their entirety to the extent not inconsistent with the present description.

1. Polymorph Form A of N—((S)-3-amino-1-(hydroxyamino)-3-methyl-1-oxobutan-2-yl)-4-(((1R,2R)-2-(hydroxymethyl)cyclopropyl)buta-1,3-diynyl)benzamide.

2. The polymorph Form A of claim 1 wherein the polymorph exhibits three predominant endotherm peaks at about 87° C., about 115° C. and about 124° C. as measured by a Differential Scanning Calorimeter (DSC) at a scan rate of 10° C. per minute.

3. The polymorph Form A of claim 1 wherein the polymorph exhibits an X-ray powder diffraction pattern having a characteristic peak expressed in degrees 2θ (+/−0.20°θ) at 5.1.

4. The polymorph Form A of claim 3 wherein the polymorph exhibits an additional characteristic peak expressed in degrees 2θ (+/−0.20°θ) at 5.5.

5. The polymorph Form A of claim 3 wherein the polymorph exhibits an additional characteristic peak expressed in degrees 2θ (+/−0.20°θ) at 6.3.

6. A pharmaceutical composition comprising a pharmaceutically acceptable carrier or diluent and polymorph Form A of N—((S)-3-amino-1-(hydroxyamino)-3-methyl-1-oxobutan-2-yl)-4-(((1R,2R)-2-(hydroxymethyl)cyclopropyl)buta-1,3-diynyl)benzamide.

7. A method for treating a subject having a bacterial infection comprising administering to a subject in need thereof a therapeutically effective amount of polymorph Form A of N—((S)-3-amino-1-(hydroxyamino)-3-methyl-1-oxobutan-2-yl)-4-(((1R,2R)-2-(hydroxymethyl)cyclopropyl)buta-1,3-diynyl)benzamide.

8. The method according to claim 7, wherein said bacterial infection is a gram-negative bacterial infection.

9. The method according to claim 8, wherein said gram-negative bacterial infection is Pseudomonas aeruginosa, Stenotrophomonas maltophila, Burkholderia cepacia, Alcaligenes xylosoxidans, or a Enterobacteriaceae, Haemophilus, Franciscellaceae or Neisseria species.

10. The method of claim 9, wherein said gram-negative bacteria is a member of the Enterobacteriaceae selected from the group consisting of Serratia, Proteus, Klebsiella, Enterobacter, Citrobacter, Salmonella, Providencia, Yersinia, Morganella, Cedecea, Edwardsiella and Escherichia.

11. A method of inhibiting a deacetylase enzyme in gram-negative bacteria comprising administering to a subject in need of such inhibition polymorph Form A of N—((S)-3-amino-1-(hydroxyamino)-3-methyl-1-oxobutan-2-yl)-4-(((1R,2R)-2-(hydroxymethyl)cyclopropyl)buta-1,3-diynyl)benzamide.

12. The method of claim 11, wherein the gram-negative bacteria are Pseudomonas aeruginosa, Stenotrophomonas maltophila, Burkholderia cepacia, Alcaligenes xylosoxidans, or a Enterobacteriaceae, Haemophilus, Franciscellaceae, or Neisseria species.

13. A method of inhibiting LpxC comprising administering to a subject in need of such inhibition an effective amount of polymorph Form A of N—((S)-3-amino-1-(hydroxyamino)-3-methyl-1-oxobutan-2-yl)-4-(((1R,2R)-2-(hydroxymethyl)cyclopropyl)buta-1,3-diynyl)benzamide.

14. Polymorph Form B of N—((S)-3-amino-1-(hydroxyamino)-3-methyl-1-oxobutan-2-yl)-4-(((1R,2R)-2-(hydroxymethyl)cyclopropyl)buta-1,3-diynyl)benzamide.

15. The polymorph Form B of claim 14 wherein the polymorph exhibits a predominant endotherm peak at about 168° C. as measured by a Differential Scanning Calorimeter (DSC) at a scan rate of 10° C. per minute.

16. The polymorph Form B of claim 14 wherein the polymorph exhibits an X-ray powder diffraction pattern having a characteristic peak expressed in degrees 2θ (+/−0.20°θ) at 22.3.

17. The polymorph Form B of claim 16 wherein the polymorph exhibits an additional characteristic peak expressed in degrees 2θ (+/−0.20°θ) at 20.0.

18. The polymorph Form B of claim 17 wherein the polymorph exhibits an additional characteristic peak expressed in degrees 2θ (+/−0.20°θ) at 21.1.

19. The polymorph Form B of claim 18 wherein the polymorph exhibits an additional characteristic peak expressed in degrees 2θ (+/−0.20°θ) at 24.9.

20. The polymorph Form B of claim 19 wherein the polymorph exhibits an additional characteristic peak expressed in degrees 2θ (+/−0.2006) at 18.0.

21. A pharmaceutical composition comprising a pharmaceutically acceptable carrier or diluent and polymorph Form B of N—((S)-3-amino-1-(hydroxyamino)-3-methyl-1-oxobutan-2-yl)-4-(((1R,2R)-2-(hydroxymethyl)cyclopropyl)buta-1,3-diynyl)benzamide.

22. A pharmaceutical composition according to claim 21, wherein said polymorph exhibits an X-ray powder diffraction pattern having a characteristic peaks expressed in degrees 2θ (+/−0.20° 0) at 22.3, 20.0 and 21.1.

23. A method for treating a subject having a bacterial infection comprising administering to a subject in need thereof a therapeutically effective amount of polymorph Form B of N—((S)-3-amino-1-(hydroxyamino)-3-methyl-1-oxobutan-2-yl)-4-(((1R,2R)-2-(hydroxymethyl)cyclopropyl)buta-1,3-diynyl)benzamide.

24. The method according to claim 23, wherein said polymorph exhibits an X-ray powder diffraction pattern having a characteristic peaks expressed in degrees 2θ (+/−0.20°θ) at 22.3, 20.0 and 21.1.

25. The method according to claim 23, wherein said bacterial infection is a gram-negative bacterial infection.

26. The method according to claim 25, wherein said gram-negative bacterial infection is Pseudomonas aeruginosa, Stenotrophomonas maltophila, Burkholderia cepacia, Alcaligenes xylosoxidans, or a Enterobacteriaceae, Haemophilus, Franciscellaceae or Neisseria species.

27. The method of claim 26, wherein said gram-negative bacteria is a member of the Enterobacteriaceae selected from the group consisting of Serratia, Proteus, Klebsiella, Enterobacter, Citrobacter, Salmonella, Providencia, Yersinia, Morganella, Cedecea, Edwardsiella and Escherichia.

28. A method of inhibiting a deacetylase enzyme in gram-negative bacteria comprising administering to a subject in need of such inhibition polymorph Form B of N—((S)-3-amino-1-(hydroxyamino)-3-methyl-1-oxobutan-2-yl)-4-(((1R,2R)-2-(hydroxymethyl)cyclopropyl)buta-1,3-diynyl)benzamide.

29. The method according to claim 28, wherein said polymorph exhibits an X-ray powder diffraction pattern having a characteristic peaks expressed in degrees 2θ (+/−0.20°θ) at 22.3, 20.0 and 21.1.

30. The method of claim 28, wherein the gram-negative bacteria are Pseudomonas aeruginosa, Stenotrophomonas maltophila, Burkholderia cepacia, Alcaligenes xylosoxidans, or a Enterobacteriaceae, Haemophilus, Franciscellaceae, or Neisseria species.

31. A method of inhibiting LpxC comprising administering to a subject in need of such inhibition an effective amount of polymorph Form B of N—((S)-3-amino-1-(hydroxyamino)-3-methyl-1-oxobutan-2-yl)-4-(((1R,2R)-2-(hydroxymethyl)cyclopropyl)buta-1,3-diynyl)benzamide.

32. The method according to claim 31, wherein said polymorph exhibits an X-ray powder diffraction pattern having a characteristic peaks expressed in degrees 2θ (+/−0.20°θ) at 22.3, 20.0 and 21.1.
